(19) United States
(12) Patent Application Publication
(10) Pub. No.: US 2020/0345423 A1
(43) Pub. Date: Nov. 5, 2020

(54) System and Method for Image-Guided Treatment Planning

(71) Applicant: Mayo Foundation for Medical Education and Research, Rochester, MN (US)

(72) Inventors: Eric E. Williamson, Rochester, MN (US); Shuai Leng, Rochester, MN (US)

(21) Appl. No.: 16/962,022

(22) PCT Filed: Jan. 15, 2019
(86) PCT No.: PCT/US2019/013687

§ 371 Date: Jul. 14, 2020

Related U.S. Application Data
(60) Provisional application No. 62/617,343, filed on Jan. 15, 2018.

(51) Int. Cl. A61B 34/10; G16H 30/20; G16H 30/40; G16H 50/20; G16H 50/30; G16H 15/00; G16H 20/40; A61B 6/03; A61B 6/00; G06T 7/00

(52) U.S. Cl. CPC A61B 34/10; G16H 30/20; G16H 30/40; G16H 50/20; G16H 50/30; G06Q 50/04; G16H 20/40; A61B 6/032; A61B 6/503; G06T 7/0012; G16H 15/00

(57) ABSTRACT

A system and method for image guided treatment planning utilizing advanced imaging techniques, including multiphase CT scanning, is disclosed. Included is a method for automatically generating a treatment report having the steps of acquiring image data from a patient, extracting patient-specific parameters from the image, analyzing the patient-specific parameters, and generating a report indicating a desired treatment. Treatment recommendations are tailored to each patient.

### CROSS-REFERENCE TO RELATED APPLICATIONS

This application claims the benefit of U.S. Provisional Patent Application Ser. No. 62/617,343, filed on Jan. 15, 2018, and entitled “SYSTEM AND METHOD FOR IMAGE GUIDED TREATMENT PLANNING.”

### BACKGROUND

Mitral regurgitation is a general term that comprises a wide range of diseases. Taken together, these diseases are an important cause of cardiac morbidity and mortality in the US and around the world. All causes of mitral regurgitation lead to disruption of the normal configuration of the mitral annulus. Additionally, mitral annular geometry is known to be of critical importance for adequate mitral valve function. Although it is widely accepted that correction of mitral regurgitation requires stabilization of the mitral annulus, conventional techniques require an open surgical procedure. Despite considerable effort, minimally invasive, catheter-based techniques for mitral annulus repair have not yet reached widespread adoption.

One reason catheter therapies for mitral annulus reconstruction have lagged is that current imaging techniques are inadequate. Catheter therapies require accurate pre-procedural measurements to ensure appropriate device sizing. The mitral annulus is a complex, three-dimensional structure which undergoes significant mechanical deformation during normal cardiac motion. The most common methods for imaging the heart are two-dimensional (conventional echocardiography) and/or static (single phase CT). Neither of these techniques is adequate to capture the dynamic geometry of the mitral annulus—particularly as that geometry becomes altered by various disease states.

Currently, two-dimensional echocardiography is primarily used to diagnose diseases of the mitral valve while single phase CT is used to size mitral devices and assess routes of device deployment. No single imaging technique has emerged to combine the dynamic and three-dimensional elements necessary to characterize the wide range of mitral diseases. Three-dimensional echocardiography is a promising technique, but is not viewed as a reliable method for obtaining precise cardiac measurements. ECG-gated CT has similar promise, but has been limited in its adoption due to the perceived high radiation dose and risks of intravenous contrast material required to obtain diagnostic cardiac images.

This lack of an agreed upon imaging standard for the assessment of dynamic mitral annular function has hindered the development of catheter deployed mitral valve therapies. Instead of focusing on the underlying anatomic abnormality that defines a specific disease state, the development of devices for mitral valve intervention has followed a “one size fits all” approach. Individual mitral valve devices have generally been employed across the broad range of mitral diseases rather than being targeted to a specific underlying mitral annulus abnormality. As such, failure rates for catheter-based mitral valve therapies have been high.

### SUMMARY OF THE DISCLOSURE

The present disclosure overcomes the drawbacks of previous systems and methods by facilitating patient-specific treatment, such as for mitral valve diseases. The systems and methods of the present disclosure allow for the analysis and the characterization of the mitral annulus and its supporting structure using images to thereby design a patient-specific treatment. In some instances, the patient-specific treatment may utilize a patient-specific device, such as replacement valve that is specifically designed for the patient, such as based on the images used for designing the treatment or other images.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1A is a perspective view of a CT imaging system configured for operation in accordance with the present disclosure.

FIG. 1B is a block diagram of a control system of the CT imaging system of FIG. 1A.

FIG. 2 is a flow chart setting forth some non-limiting examples steps of a method for using an imaging system to select an appropriate treatment in accordance with the present disclosure.

FIG. 3 is a diagram illustrating a workflow for generating treatment plan data based on processing and analyzing patient metric data generated from imaging data.

FIG. 4 is a diagram illustrating treatments for mitral valve disorders in accordance with some embodiments of the systems and methods described in the present disclosure.

FIG. 5 is a block diagram of an example system that can implement the methods described in the present disclosure.

FIG. 6 is a block diagram illustrating examples of hardware components that can implement the system of FIG. 5.

FIG. 7 is a schematic illustration of an example graphical user interface (“GUI”) generated by a system for selecting the contours of the mitral valve annulus.

FIG. 8 is a schematic illustration of an example GUI generated by a system for adjusting mitral valve annulus contours.

FIG. 9 is another schematic illustration of an example GUI generated by a system for adjusting mitral valve annulus contours.

FIG. 10 is yet another schematic illustration of an example GUI generated by a system for adjusting mitral valve annulus contours.

FIGS. 11A and 11B illustrates example of incorrectly positioned contour points (FIG. 11A) and correctly positioned contour points after adjustment (FIG. 11B).

FIG. 12 is a schematic illustration of an example GUI generated by a system for selecting landmarks on the mitral annulus.

FIG. 13 is a schematic illustration of an example GUI generated by a system for adjusting the landmarks.

FIG. 14 is a schematic illustration of an example GUI generated by a system for selecting the tips of the papillary muscles.

FIG. 15 is a schematic illustration of an example GUI generated by a system for displaying or otherwise extracting patient-specific parameters from the imaging data.

FIG. 16 is a schematic illustration of a system for generating a report based on the analysis of the patient-specific parameters.

FIG. 17 illustrates an example of example analyzing the gap between a deployed prosthesis and the mitral annulus, which indicates how much potential leakage may occur after the prosthesis deployment.

### DETAILED DESCRIPTION

Described here are systems and methods for characterization of mitral structure and function to guide interventional procedures, such as transcatheter valve implantation. The systems and methods include acquiring and processing imaging data of a patient in order to generate patient metric data that indicates patient specific anatomy relevant for planning a treatment. For instance, the patient specific metric may include quantitative measurements of patient-specific anatomy, such as measurements associated with a mitral valve. Treatment plan data are generated by processing or otherwise analyzing these patient metric data. The treatment plan data can include an indication of a particular treatment option for the patient that is optimal based on the patient-specific anatomy. The treatment plan data can also include data associated with prostheses, devices, or instruments that can be used in the optimal treatment plan option. For instance, the treatment plan data may include data describing an optimal prosthesis for use in a treatment plan.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The disclosed subject matter may be implemented as a system, method, apparatus, or article of manufacture using standard programming and/or engineering techniques and/or programming to produce hardware, firmware, software, or any combination thereof to implement aspects detailed herein.

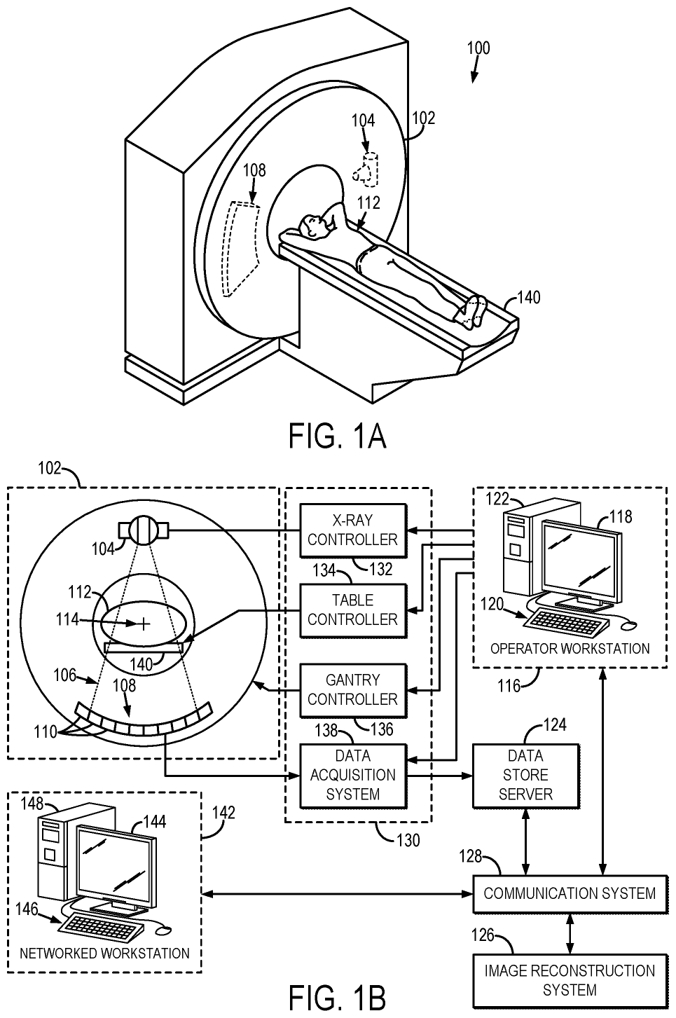

Referring particularly now to FIGS. 1A and 1B, an example of an x-ray computed tomography (“CT”) imaging system 100 is illustrated. The CT system includes a gantry 102, to which at least one x-ray source 104 is coupled. The x-ray source 104 projects an x-ray beam 106, which may be a fan-beam or cone-beam of x-rays, towards a detector array 108 on the opposite side of the gantry 102. The detector array 108 includes a number of x-ray detector elements 110. Together, the x-ray detector elements 110 sense the projected x-rays 106 that pass through a subject 112, such as a medical patient or an object undergoing examination, that is positioned in the CT system 100. Each x-ray detector element 110 produces an electrical signal that may represent the intensity of an impinging x-ray beam and, hence, the attenuation of the beam as it passes through the subject 112. In some configurations, each x-ray detector 110 is capable of counting the number of x-ray photons that impinge upon the detector 110. During a scan to acquire x-ray projection data, the gantry 102 and the components mounted thereon rotate about a center of rotation 114 located within the CT system 100.

The CT system 100 also includes an operator workstation 116, which typically includes a display 118; one or more input devices 120, such as a keyboard and mouse; and a computer processor 122. The computer processor 122 may include a commercially available programmable machine running a commercially available operating system. The operator workstation 116 provides the operator interface that enables scanning control parameters to be entered into the CT system 100. In general, the operator workstation 116 is in communication with a data store server 124 and an image reconstruction system 126. By way of example, the operator workstation 116, data store sever 124, and image reconstruction system 126 may be connected via a communication system 128, which may include any suitable network connection, whether wired, wireless, or a combination of both. As an example, the communication system 128 may include both proprietary or dedicated networks, as well as open networks, such as the internet.

The operator workstation 116 is also in communication with a control system 130 that controls operation of the CT system 100. The control system 130 generally includes an x-ray controller 132, a table controller 134, a gantry controller 136, and a data acquisition system 138. The x-ray controller 132 provides power and timing signals to the x-ray source 104 and the gantry controller 136 controls the rotational speed and position of the gantry 102. The table controller 134 controls a table 140 to position the subject 112 in the gantry 102 of the CT system 100.

The DAS 138 samples data from the detector elements 110 and converts the data to digital signals for subsequent processing. For instance, digitized x-ray data is communicated from the DAS 138 to the data store server 124. The image reconstruction system 126 then retrieves the x-ray data from the data store server 124 and reconstructs an image therefrom. The image reconstruction system 126 may include a commercially available computer processor, or may be a highly parallel computer architecture, such as a system that includes multiple-core processors and massively parallel, high-density computing devices. Optionally, image reconstruction can also be performed on the processor 122 in the operator workstation 116. Reconstructed images can then be communicated back to the data store server 124 for storage or to the operator workstation 116 to be displayed to the operator or clinician.

The CT system 100 may also include one or more networked workstations 142. By way of example, a networked workstation 142 may include a display 144; one or more input devices 146, such as a keyboard and mouse; and a processor 148. The networked workstation 142 may be located within the same facility as the operator workstation 116, or in a different facility, such as a different healthcare institution or clinic.

The networked workstation 142, whether within the same facility or in a different facility as the operator workstation 116, may gain remote access to the data store server 124 and/or the image reconstruction system 126 via the communication system 128. Accordingly, multiple networked workstations 142 may have access to the data store server 124 and/or image reconstruction system 126. In this manner, x-ray data, reconstructed images, or other data may be exchanged between the data store server 124, the image reconstruction system 126, and the networked workstations 142, such that the data or images may be remotely processed by a networked workstation 142. This data may be exchanged in any suitable format, such as in accordance with the transmission control protocol (“TCP”), the internet protocol (“IP”), or other known or suitable protocols.

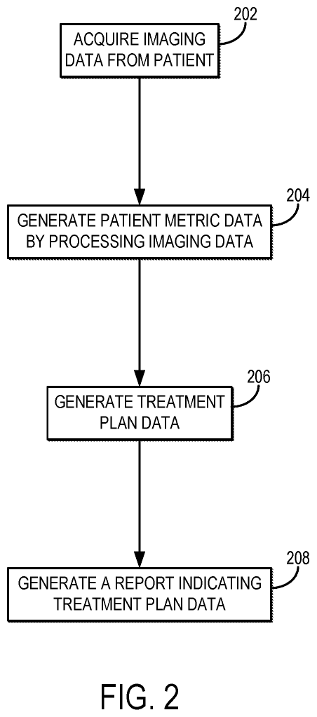

Referring to FIG. 2, a process 200 for selecting the appropriate treatment based on image guidance is illustrated. The treatment, as one non-limiting example, may be to treat a mitral regurgitation disorder in a patient-specific manner. In such examples, the methods described in the present disclosure may provide for the selection of a patient-specific treatment option based on the anatomy of the patient as determined via images (e.g., images showing a patient's mitral annulus). As will be described, the treatment may include a transcatheter treatment.

At process block 202, imaging data may be acquired from the patient or previously acquired imaging data may be provided to a computer system for processing. For example, this process may be accomplished using a multiphase CT scan, which may be performed using the above-described CT system. However, other imaging techniques and modalities may be used, including magnetic resonance imaging (“MRI”) or echocardiography.

Once imaging data of the patient have been acquired or otherwise provided to a computer system for processing, patient metric data are generated by processing the imaging data at process block 204. The patient metric data include patient-specific metrics that are extracted, computed, measured, or otherwise generated from the imaging data. For example, in the case of planning treatment for cardiac or mitral valve disorders, the patient metric data may include a minimum circumference of the mitral annulus or the maximum circumference of the mitral annulus determined from the imaging data. In other non-limiting examples, the patient metric data may include an intercommissural (“IC”) distance, a septal-to-lateral (“SL”) distance, or both.

As one non-limiting example, when the methods described in the present disclosure are implemented for analyzing cardiac or mitral valve disorders, generating the patient metric data may include computing a variation in the circumference of the mitral annulus based on the imaging data, for example, as follows:

$$V = \frac{( {C}_{ma x} - {C}_{m i n} )}{( {C}_{ma x} + {C}_{m i n} )} ; \qquad ( 1 )$$

where Cₘₐₓ is the maximum circumference of the mitral annulus, Cₘᵢₙ is the minimum circumference of the mitral annulus, and V is the variation in the circumference of the mitral annulus. This variation indicates or otherwise estimates the variation in the circumference of the mitral annulus during the cardiac cycle, or portion thereof, as depicted or otherwise represented in the imaging data.

Additionally or alternatively, generating the patient metric data may include measuring or otherwise calculating the IC distance, the SL distance, or both. In these instances, generating the patient metric data may also include calculating a ratio between the IC distance and SL distance (referred to as an “ISR”), as follows:

$$I S R = \frac{IC distance}{SL distance} . \qquad ( 2 )$$

Additionally or alternatively, the patient metric data may include an annulus circumference, an anteroposterior diameter, an anterolateral-posteromedial diameter, an annulus ellipticity, annulus height, planar surface area, distance between papillary muscle heads, anterolateral papillary muscle distance, and posteromedial papillary muscle distance.

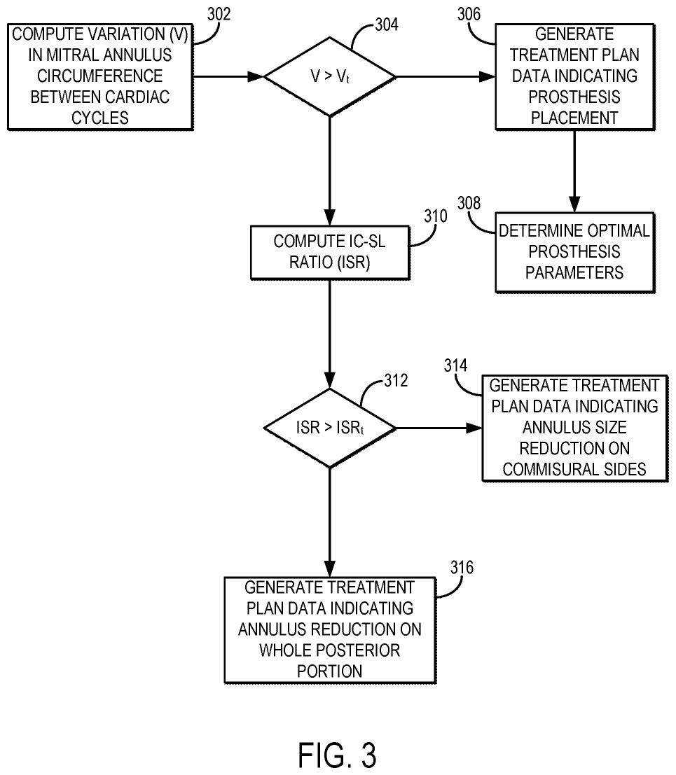

At process block 206, a treatment plan may be generated based at least in part on the imaging data, the patient metric data, or both. The treatment plan can include data indicated a treatment option or choice determined in part on analyzing the imaging data, the patient metric data, or both. An example workflow for analyzing the imaging data, patient metric data, or both, to generate a treatment plan is illustrated in FIG. 3.

At process block 208, the treatment plan data generated as a result of the algorithmic analysis of the imaging data and patient metric data are synthesized to generate a report indicating one or more potential treatment options. Generating the report may include generating one or more display element from the treatment plan data and displaying the display elements on a graphical user interface or other display. For instance, the generated report may include a graphical user interface that displays an indication of an optimal treatment option for the patient. In other instances, the report may include textual information or other data indicating or otherwise representing the optimal treatment option for the patient. The generated report may be displayed to a user, or stored for later use or retrieval, such as being stored as a part of the patient's electronic health record.

Referring to FIG. 3, generating a treatment plan according to some embodiments described in the present disclosure can include measuring the variation in the circumference of the mitral annulus, as indicated at process block 302. At process block 304, the calculated variation in the mitral annulus circumference can be compared to a first threshold value to determine whether a prosthesis should be placed. When the criteria for the comparison are met, treatment plan data are generated at process block 306. These treatment plan data indicate that a prosthesis should be placed for the patient. The treatment plan data may include a graphic element that is generated and displayed to a user on a graphical user interface or other display. In other instances, the treatment plan data may be textual or other data that are stored in a report or other data structure for later use or retrieval.

For instance, the variation metric can be compared to the first threshold value and when the variation is above the first threshold value, treatment plan data are generated that indicate that a given commercially-available replacement valve may be appropriate for deployment within the specific anatomy of the patient. The first threshold value may have a predetermined value. For example, the first threshold value can be determined from a database of patients with normal mitral valves. Based on heart size data and mitral annulus size data stored in such a database, the first threshold value can be selected or dynamically generated.

When the determination is made that the patient-specific treatment plan should indicate deploying a mitral valve prosthesis, the imaging data, the patient metric data, or both, can be further analyzed to determine a mitral annulus prosthesis that optimally matches the patient-specific anatomy, as indicated at process block 308. For instance, a match can be found from the same database from which the first threshold value is determined. The optimal match can be based on the mitral annulus data in the database that provides the most similar heart size to the patient-specific anatomy. For instance, the annulus of the matched case in the database can be used as the restored size to select the appropriate prosthesis.

In some embodiments, the heart size can be defined as a function of left ventricle volume, left atrium volume, and left ventricle myocardium mass: f(V_{LV}, V_{LA}, M_{LV}) The objective then is to select the annulus size to minimize the difference, i.e. min ∥fₚₐₜᵢₑₙₜ−fₙₒᵣₘₐₗ∥.

To assess the fit of prosthesis on each annulus, measurements of linear distance (1D), surface area (2D), volume (3D), or combinations thereof, of the gaps between the prosthesis and mitral annulus can used. For any given point on the annulus, linear distance can be calculated as the shortest distance between this point and any point on the prosthesis. Surface area can be calculated as the plane perpendicular to the main axis of the prosthesis. Volume can be calculated as the total volume of gaps within the range of the intersection between prosthesis and annulus. Mean and maximal values of linear distance and surface area can be stored in the treatment plan data, displayed to a user (e.g., via a graphical user interface), or otherwise reported.

When the criteria for comparing the variation to the first threshold are not satisfied, the ISR values computed in the patient metric data, as indicated at step 310, can be compared to a second threshold value, as indicated at step 312. Like the first threshold value, the second threshold value may be a predetermined value. In some instances, the second threshold value can be determined, computed, or otherwise based on normal mitral annulus data, heart size data, or both, which are stored in a database.

When the criteria for comparing the ISR to the second threshold are satisfied, the treatment plan data can be generated to include an indication that the patient can be treated by reducing the distance of the mitral annulus along the IC direction to restore the ellipticity of the annulus, as indicated at process block 314.

When the criteria for comparing the ISR to the second threshold are not satisfied, the treatment plan data can be generated to include an indication that the patient can be treated by reducing the posterior side of the mitral annulus, as indicated at process block 316.

Thus, as one non-limiting example, if a patient has variation higher than a predetermined threshold, V>Vₜ, the generated treatment plan data can indicate that a mitral valve prosthesis should be placed for the patient. Otherwise, if V<Vₜ and the ISR is higher than a predetermined threshold, ISR>ISRₜ, the generated treatment plan data can indicate that the patient can be treated with reduction of annulus mainly around the commissures to reduce the distance along the IC direction and to restore the ellipticity of the annulus. This treatment can be achieved, for instance, using anchors or other instruments to reduce the annulus. Alternatively, if V<Vₜ, the generated treatment plan data may indicate that a different mitral valve prosthesis should be used than if V>Vₜ. Otherwise, if V<Vₜ and ISR<ISRₜ, the generated treatment plan data can indicate that the patient should be treated with reduction of the whole posterior portion of the annulus. This treatment could be achieved, for instance, with a constraining band or other suitable instruments.

Within the context of this non-limiting example, for patients whose patient-specific treatment plan data indicate that a mitral valve prosthesis should be placed, the selection of that prosthesis can be based on the above criteria. The goal of the prosthesis is to restore the mitral geometry so that optimal heart function can be achieved. Intuitively, one may want to select the prosthesis that restores the annulus back to the size before the onset of mitral disease. This approach may have limitations due to at least two factors. First, the size of the annulus of a particular patient before mitral disease may not be available in most cases. Second, as the heart remodels during the progress of mitral valve disease, the size of the patient's heart will not be the same as before disease onset. As such, restoring the annulus to the pre-disease size may not be optimal based on the changes in the heart size.

Continuing with the above-described non-limiting example, by determining the variation in the circumference of the mitral annulus and comparing the calculation against the first threshold value, the process yields an indication of an appropriate treatment, such as a desired size, brand, or the like of mitral valve prosthesis. For example, if the variation in the circumference of the mitral annulus is greater than the first threshold value, a first valve size or brand may be indicated as part of the report on treatment options. On the other hand, if the variation in the circumference of the mitral annulus is less than the first threshold value, a second valve size or brand may be indicated. As another example, if the variation in the circumference of the mitral annulus is less than the first threshold value, further non-prosthetic treatment or further analysis may be provided as part of the treatment options. If further analysis is desired, the calculation of the ISR may be part of the subsequent steps.

Specifically, the ISR may be determined as one of the extracted patient metrics and compared against the second threshold value, such that the step of selecting an appropriate treatment may readily include selecting the reduction of the annulus as the treatment if the ISR is less than the second threshold value. If the ISR is greater than the second threshold value, the appropriate treatment may be a reduction of the posterior portion of the mitral annulus. This information and additional information may be communicated automatically via the report at process block 208.

Treatments selected may be accomplished with a variety of specific procedures. If a prosthesis has been selected as the treatment, custom designed prosthetic valves may be utilized. For example, the report produced at process block 208 may include parameters for creating a custom prosthetic, for example, via additive manufacturing process/three-dimensional printing. In such instances, the treatment plan data may also include instructions or models for an additive manufacturing process. The prosthetic devices may be implemented in a transcatheter procedure or in other more invasive procedures. In some circumstances, a reduction of the annulus can be accomplished using anchors, however other instruments that accomplish this goal can be utilized and indicated in the report generated in process block 208. A reduction in the posterior of the annulus can be achieved with a constraining band, however, other instruments which accomplish this goal may also be implemented and indicated in the report generated in process block 208.

The report generated at process block 208 may include instructions for device, including prosthetic, development. For example, the report may include parameters for additive or 3D printing techniques to construct patient-specific heart models of mitral valve disease and fluid dynamics for simulation of valve prostheses deployment. Thus, patient-specific models of mitral valve disease, which can be used to assess in vitro fit of valve prostheses, may be developed using the above-described systems and methods. Mitral valve prostheses can be deployed in these patient-specific models and initial size match can be assessed. This allows appropriate sizing of valve prostheses for different mitral disease.

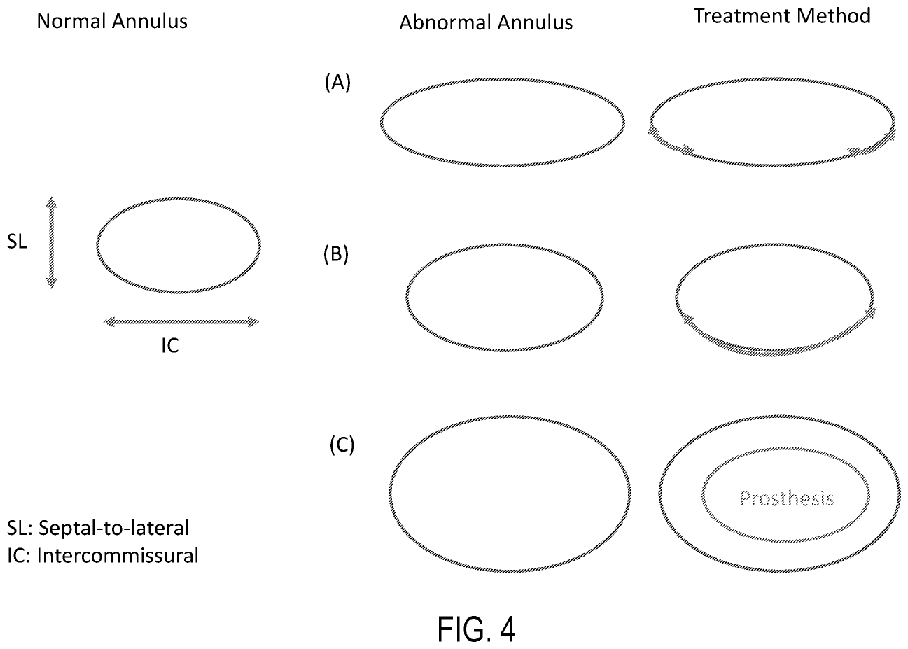

FIG. 4 illustrates examples of a normal mitral annulus in relation to different examples of an abnormal mitral annulus. Different treatment methods selected for the different abnormalities are also illustrated in FIG. 4. For instance, in Example (A), analysis of patient metric data results in treatment plan data indicating that the mitral annulus should be reduced on the commissural sides, in Example (B) analysis of patient metric data results in treatment plan data indicating that the posterior portion of the mitral annulus should be reduced, and in Example (C) analysis of patient metric data results in treatment plan data indicating that a prosthesis should be deployed or otherwise placed.

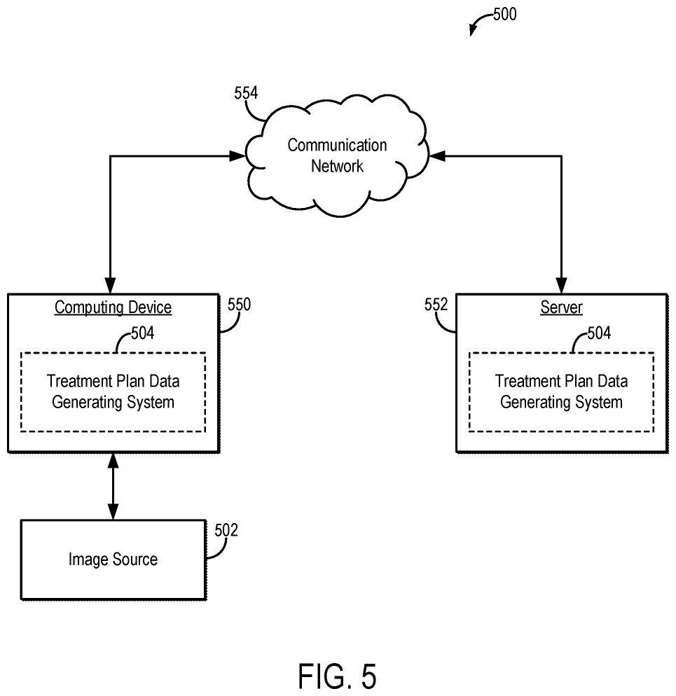

Referring now to FIG. 5, an example of a system 500 for generating treatment plan data in accordance with some embodiments of the systems and methods described in the present disclosure is shown. As shown in FIG. 5, a computing device 550 can receive one or more types of data (e.g., imaging data, patient metric data) from image source 502. In some embodiments, computing device 550 can execute at least a portion of a treatment plan data generating system 504 to generate treatment plan data from imaging data received from the image source 502.

Additionally or alternatively, in some embodiments, the computing device 550 can communicate information about data received from the image source 502 to a server 552 over a communication network 554, which can execute at least a portion of the treatment plan data generating system 504 to generate treatment plan data from imaging data received from the image source 502. In such embodiments, the server 552 can return information to the computing device 550 (and/or any other suitable computing device) indicative of an output of the treatment plan data generating system 504 to generate treatment plan data from imaging data received from the image source 502.

In some embodiments, computing device 550 and/or server 552 can be any suitable computing device or combination of devices, such as a desktop computer, a laptop computer, a smartphone, a tablet computer, a wearable computer, a server computer, a virtual machine being executed by a physical computing device, and so on. The computing device 550 and/or server 552 can also reconstruct images from the data.

In some embodiments, image source 502 can be any suitable source of image data (e.g., measurement data, images reconstructed from measurement data), such as a computed tomography (“CT”) imaging system, a magnetic resonance imaging (“MRI”) system, an ultrasound imaging system (e.g., for echocardiography imaging data), another computing device (e.g., a server storing image data), and so on. In some embodiments, image source 502 can be local to computing device 550. For example, image source 502 can be incorporated with computing device 550 (e.g., computing device 550 can be configured as part of a device for capturing, scanning, and/or storing images). As another example, image source 502 can be connected to computing device 550 by a cable, a direct wireless link, and so on. Additionally or alternatively, in some embodiments, image source 502 can be located locally and/or remotely from computing device 550, and can communicate data to computing device 550 (and/or server 552) via a communication network (e.g., communication network 554).

In some embodiments, communication network 554 can be any suitable communication network or combination of communication networks. For example, communication network 554 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 108 can be a local area network, a wide area network, a public network (e.g., the Internet), a private or semi-private network (e.g., a corporate or university intranet), any other suitable type of network, or any suitable combination of networks. Communications links shown in FIG. 5 can each be any suitable communications link or combination of communications links, such as wired links, fiber optic links, Wi-Fi links, Bluetooth links, cellular links, and so on.

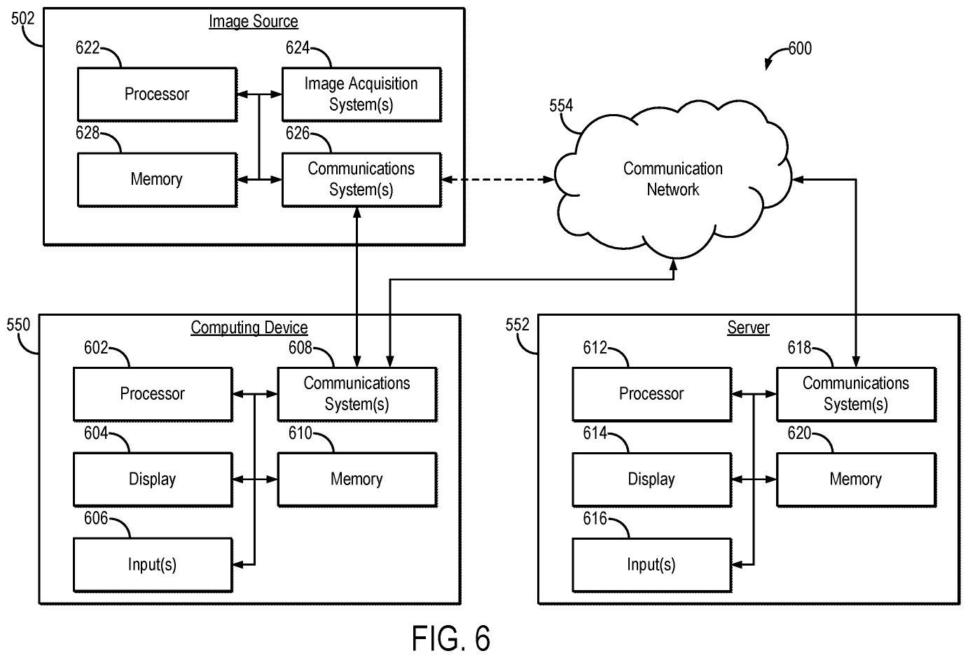

Referring now to FIG. 6, an example of hardware 600 that can be used to implement image source 502, computing device 550, and server 554 in accordance with some embodiments of the systems and methods described in the present disclosure is shown. As shown in FIG. 6, in some embodiments, computing device 550 can include a processor 602, a display 604, one or more inputs 606, one or more communication systems 608, and/or memory 610. In some embodiments, processor 602 can be any suitable hardware processor or combination of processors, such as a central processing unit (“CPU”), a graphics processing unit (“GPU”), and so on. In some embodiments, display 604 can include any suitable display devices, such as a computer monitor, a touchscreen, a television, and so on. In some embodiments, inputs 606 can include any suitable input devices and/or sensors that can be used to receive user input, such as a keyboard, a mouse, a touchscreen, a microphone, and so on.

In some embodiments, communications systems 608 can include any suitable hardware, firmware, and/or software for communicating information over communication network 554 and/or any other suitable communication networks. For example, communications systems 608 can include one or more transceivers, one or more communication chips and/or chip sets, and so on. In a more particular example, communications systems 608 can include hardware, firmware and/or software that can be used to establish a Wi-Fi connection, a Bluetooth connection, a cellular connection, an Ethernet connection, and so on.

In some embodiments, memory 610 can include any suitable storage device or devices that can be used to store instructions, values, data, or the like, that can be used, for example, by processor 602 to present content using display 604, to communicate with server 552 via communications system(s) 608, and so on. Memory 610 can include any suitable volatile memory, non-volatile memory, storage, or any suitable combination thereof. For example, memory 610 can include RAM, ROM, EEPROM, one or more flash drives, one or more hard disks, one or more solid state drives, one or more optical drives, and so on. In some embodiments, memory 610 can have encoded thereon, or otherwise stored therein, a computer program for controlling operation of computing device 550. In such embodiments, processor 602 can execute at least a portion of the computer program to present content (e.g., images, user interfaces, graphics, tables), receive content from server 552, transmit information to server 552, and so on.

In some embodiments, server 552 can include a processor 612, a display 614, one or more inputs 616, one or more communications systems 618, and/or memory 620. In some embodiments, processor 612 can be any suitable hardware processor or combination of processors, such as a CPU, a GPU, and so on. In some embodiments, display 614 can include any suitable display devices, such as a computer monitor, a touchscreen, a television, and so on. In some embodiments, inputs 616 can include any suitable input devices and/or sensors that can be used to receive user input, such as a keyboard, a mouse, a touchscreen, a microphone, and so on.

In some embodiments, communications systems 618 can include any suitable hardware, firmware, and/or software for communicating information over communication network 554 and/or any other suitable communication networks. For example, communications systems 618 can include one or more transceivers, one or more communication chips and/or chip sets, and so on. In a more particular example, communications systems 618 can include hardware, firmware and/or software that can be used to establish a Wi-Fi connection, a Bluetooth connection, a cellular connection, an Ethernet connection, and so on.

In some embodiments, memory 620 can include any suitable storage device or devices that can be used to store instructions, values, data, or the like, that can be used, for example, by processor 612 to present content using display 614, to communicate with one or more computing devices 550, and so on. Memory 620 can include any suitable volatile memory, non-volatile memory, storage, or any suitable combination thereof. For example, memory 620 can include RAM, ROM, EEPROM, one or more flash drives, one or more hard disks, one or more solid state drives, one or more optical drives, and so on. In some embodiments, memory 620 can have encoded thereon a server program for controlling operation of server 552. In such embodiments, processor 612 can execute at least a portion of the server program to transmit information and/or content (e.g., data, images, a user interface) to one or more computing devices 550, receive information and/or content from one or more computing devices 550, receive instructions from one or more devices (e.g., a personal computer, a laptop computer, a tablet computer, a smartphone), and so on.

In some embodiments, image source 502 can include a processor 622, one or more image acquisition systems 624, one or more communications systems 626, and/or memory 628. In some embodiments, processor 622 can be any suitable hardware processor or combination of processors, such as a CPU, a GPU, and so on. In some embodiments, the one or more image acquisition systems 624 are generally configured to acquire data, images, or both, and can include a CT system, and MRI system, an ultrasound system, or another suitable medical imaging system. Additionally or alternatively, in some embodiments, one or more image acquisition systems 624 can include any suitable hardware, firmware, and/or software for coupling to and/or controlling operations of a CT system, and MRI system, an ultrasound system, or other suitable medical imaging system. In some embodiments, one or more portions of the one or more image acquisition systems 624 can be removable and/or replaceable.

Note that, although not shown, image source 502 can include any suitable inputs and/or outputs. For example, image source 502 can include input devices and/or sensors that can be used to receive user input, such as a keyboard, a mouse, a touchscreen, a microphone, a trackpad, a trackball, and so on. As another example, image source 502 can include any suitable display devices, such as a computer monitor, a touchscreen, a television, etc., one or more speakers, and so on.

In some embodiments, communications systems 626 can include any suitable hardware, firmware, and/or software for communicating information to computing device 550 (and, in some embodiments, over communication network 554 and/or any other suitable communication networks). For example, communications systems 626 can include one or more transceivers, one or more communication chips and/or chip sets, and so on. In a more particular example, communications systems 626 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 628 can include any suitable storage device or devices that can be used to store instructions, values, data, or the like, that can be used, for example, by processor 622 to control the one or more image acquisition systems 624, and/or receive data from the one or more image acquisition systems 624; to images from data; present content (e.g., images, a user interface) using a display; communicate with one or more computing devices 550; and so on. Memory 628 can include any suitable volatile memory, non-volatile memory, storage, or any suitable combination thereof. For example, memory 628 can include RAM, ROM, EEPROM, one or more flash drives, one or more hard disks, one or more solid state drives, one or more optical drives, and so on. In some embodiments, memory 628 can have encoded thereon, or otherwise stored therein, a program for controlling operation of image source 502. In such embodiments, processor 622 can execute at least a portion of the program to generate images, transmit information and/or content (e.g., data, images) to one or more computing devices 550, receive information and/or content from one or more computing devices 550, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

In a non-limiting example, the systems described in the present disclosure can implement the methods described herein to generate treatment plan data and generate a report indicating those data. For instance, a computing device 550 can select a single phase from imaging data generated by a multiphase CT scan. The treatment plan data generating system 504 can then implement the mitral valve analysis described above to generate patient metric data based on that single phase. The computing device 550 and treatment plan data generating system 504 can be programmed to ensure that a single phase has loaded.

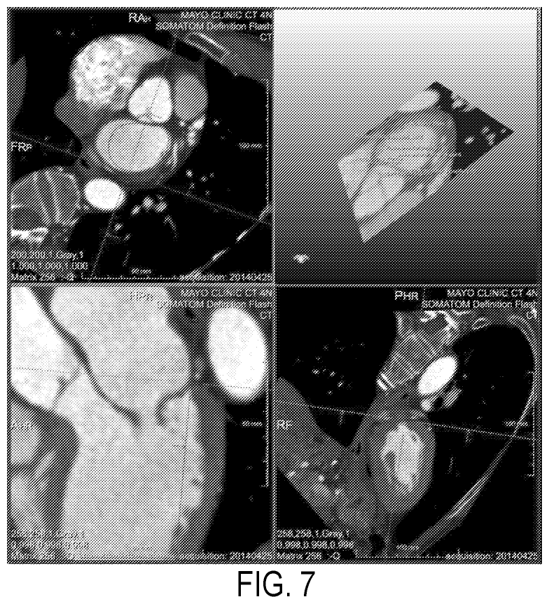

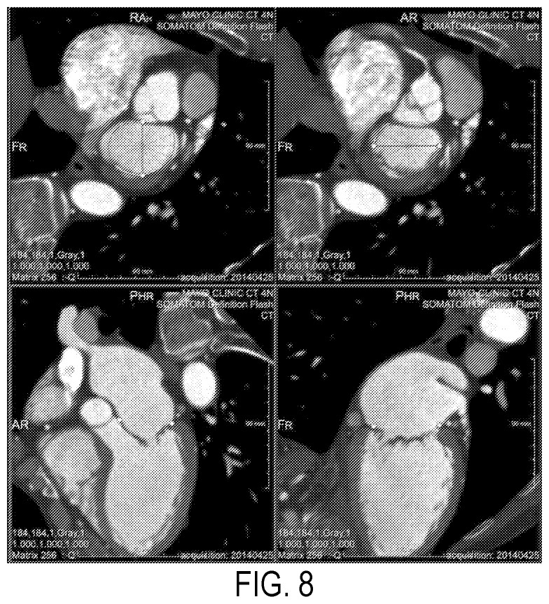

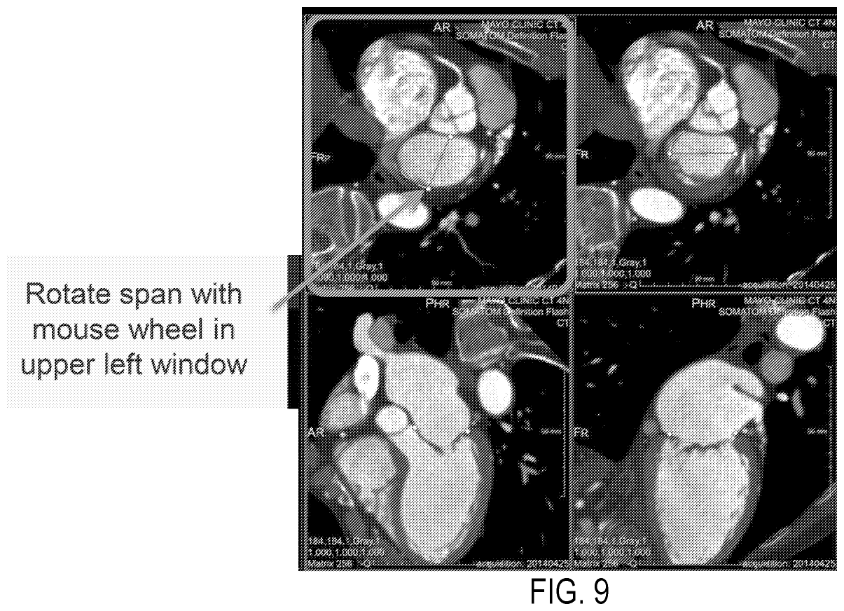

FIGS. 7-16 illustrate an example graphical user interface (“GUI”) implementing the systems and methods described in the present disclosure for generating treatment plan data from imaging data. FIG. 7 illustrates an example GUI displaying imaging data and enabling a user to generate and select contours of a mitral valve. FIGS. 8 and 9 illustrate an example GUI displaying imaging data and enabling a user to adjust the contours selected based on the imaging data. Adjusting the contours may include computing patient metric data based on the adjusted contours, and these patient metric data can be displayed or otherwise reported by the GUI.

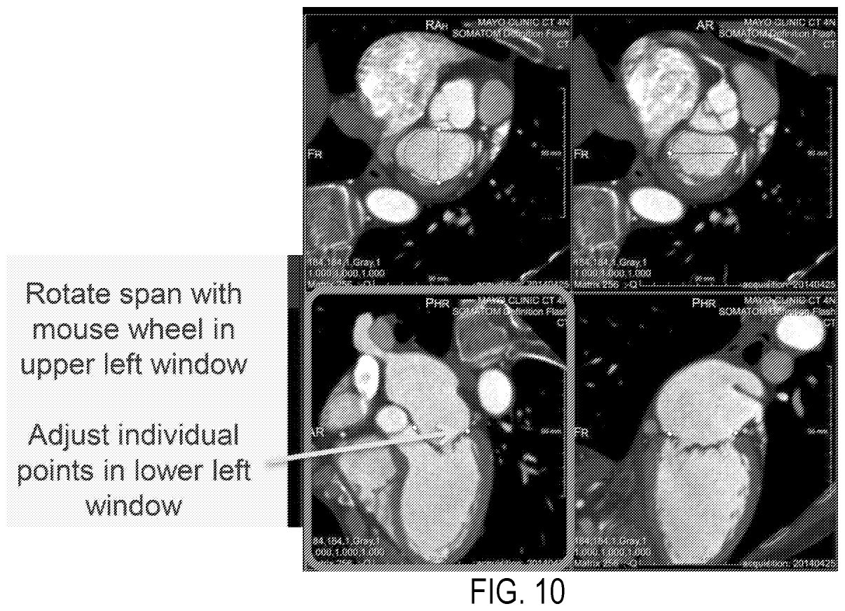

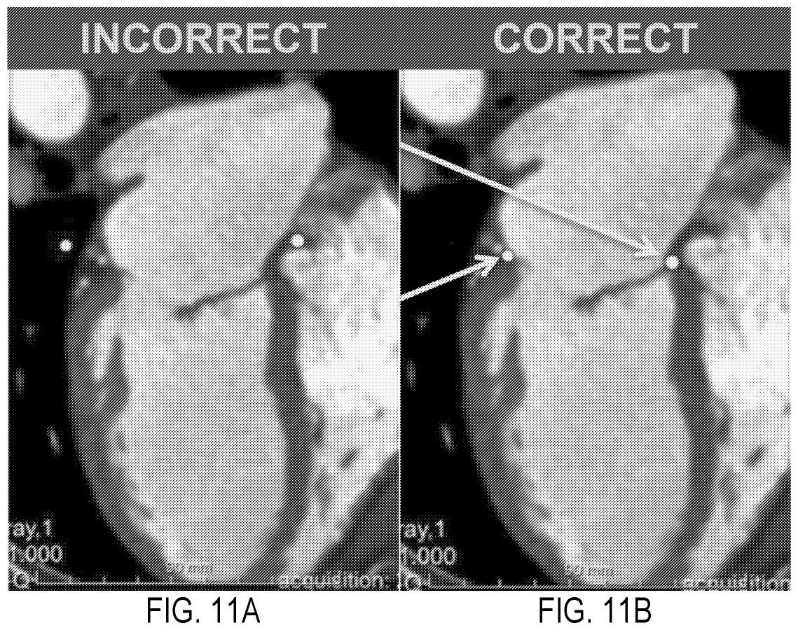

As one example, adjusting the contours may include adjusting individual points, as shown in FIG. 10 and FIGS. 11A-11B. For instance, adjusting the contours may include selecting and moving points that are displayed on the GUI. The points may be moved, for instance, to the attachment point of the adjacent valve leaflet, as shown in FIGS. 11A and 11B. If the leaflet attachment point is difficult to visualize, the selected points can also be moved to be positioned adjacent the point where the left ventricle myocardium merges with the left atrium wall. The leaflets may have a triangular form in the imaging data. In such instances, the points can be automatically or semi-automatically positioned at the apex of a triangle outside of the blood pool and at the base of the triangle inside of the blood pool. Preferably, points associated with the anterior horn should not be adjusted. In some instances, the GUI can be programmed to restrict movement of these contour points, or otherwise provide a warning to a user is these points are moved or attempted to be moved. When the anterior horn point is surrounded by contrast, however, it may be advantageous to move the contour point horizontally until it is positioned on the annulus. After the annulus contour has been adjusted a final check can be performed to verify whether any of the annulus contour points are significantly out of alignment. For instance, the annulus contour tracing should be generally shaped as a smooth oval.

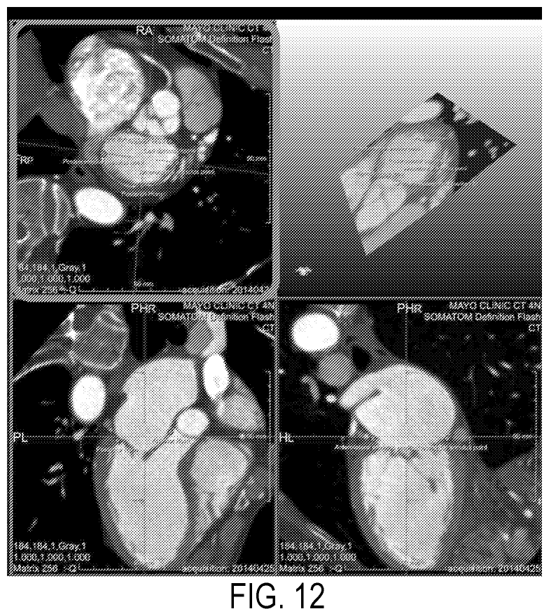

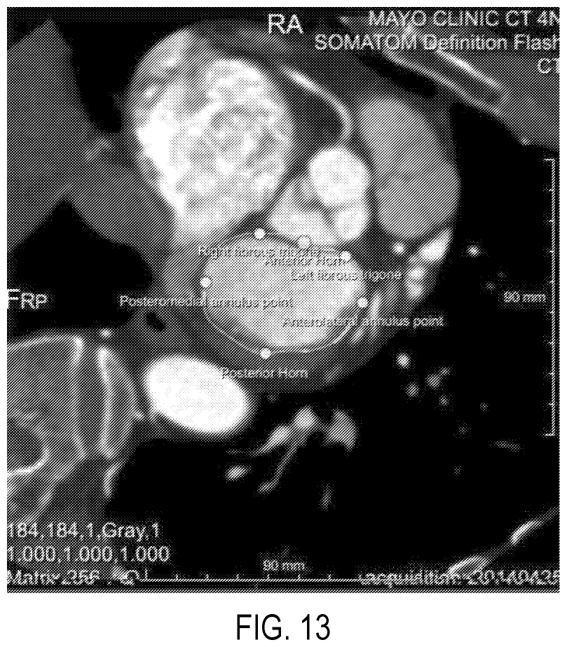

FIG. 12 illustrates an example of a GUI displaying imaging data and enabling a user to select landmarks on the mitral annulus. FIG. 13 illustrates an example of a GUI displaying imaging data and enabling a user to adjust the selected landmarks on the mitral annulus. The landmarks can be selected to correspond to specific mitral valve anatomy, such as at fibrous trigones (e.g., at the corners between the aortic and mitral valves), at the posteromedial and anterolateral annulus points (e.g., at the midpoint of the mitral annulus), and at the anterior and posterior horns (e.g., at the points bisecting the TT and IC distances).

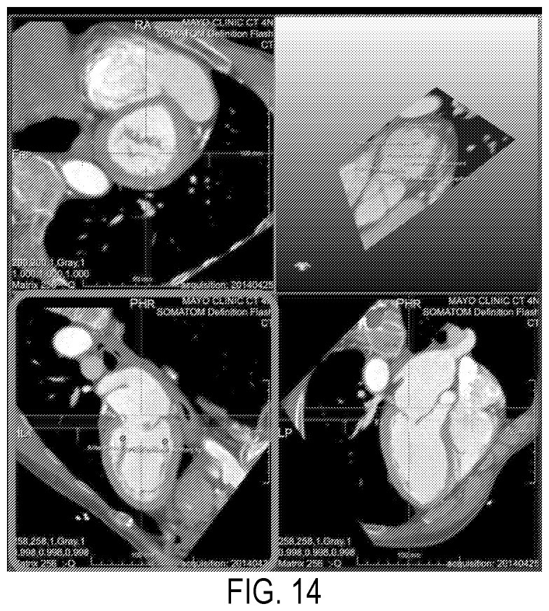

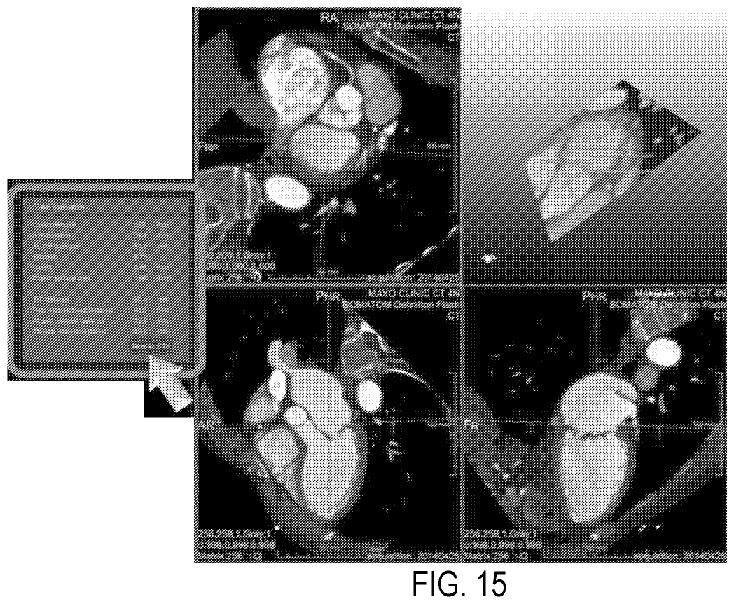

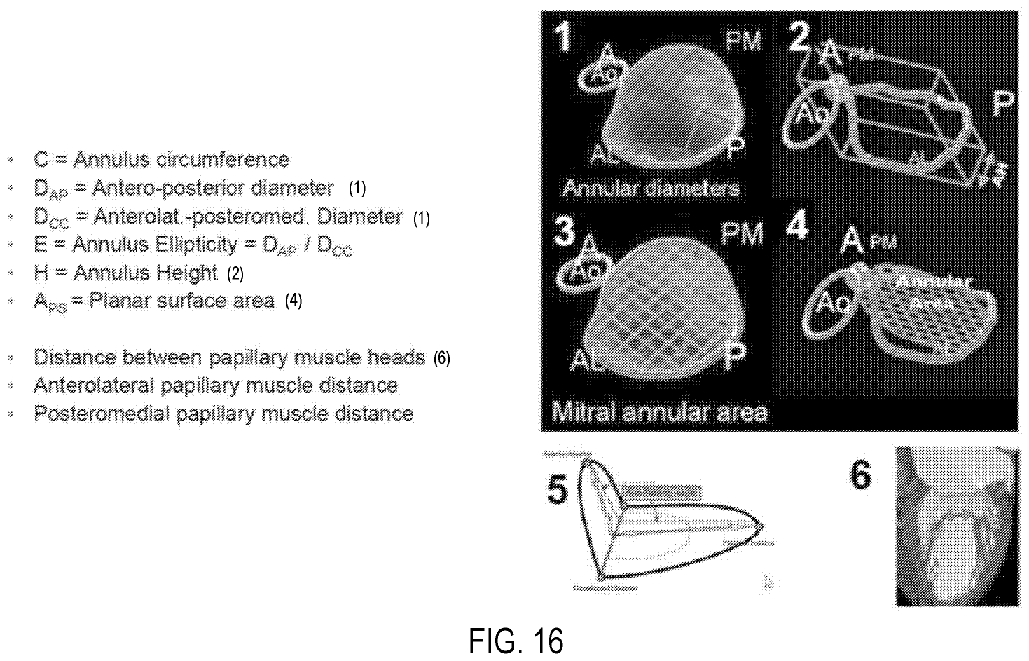

FIG. 14 illustrates an example of a GUI displaying imaging data and enabling a user to select the tips of the papillary muscles. FIG. 15 illustrates an example of a GUI displaying imaging data and reporting patient metric data computed or otherwise measured or extracted from the imaging data. FIG. 16 illustrates an example of a report generated by the computing device 550 and/or treatment plan data generating system 504, which indicates patient metric data and an analysis based on the patient-specific parameters.

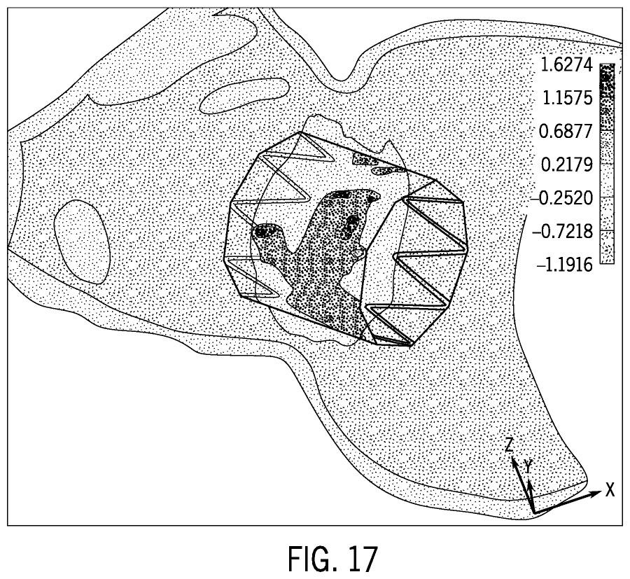

FIG. 17 illustrates and example of a GUI displaying data of measurements made after deployment of a prosthesis. After prosthesis deployment, the gap between the mitral annulus and the deployed prosthesis can be measured, estimated, or otherwise analyzed. Measuring this gap can be used to determine leakage from the left ventricle to the left atrium. As one example shown in FIG. 17, the contacting area and gap with the mitral annulus have been calculated and shown with the color map indicating distance at each point. Thus, analyzing the gap between prosthesis and mitral annulus can include acquiring imaging data from the patient after deploying the prosthesis and processing the imaging data to calculate, measure, or otherwise estimate distances between the prosthesis and surrounding anatomy (e.g., the mitral annulus). These distances can then be stored as data that are displayed in a GUI or otherwise reported to a user. This information can be used to generate updated treatment plan data, which may indicate whether the prosthesis should be repositioned or if additional treatment would be useful for addressing any leakage between the deployed prosthesis and mitral annulus.

Thus, the present disclosure provides systems, methods, and software to characterize mitral annular structure and function in patients without and with mitral valve disorders to define annular remodeling in various disease states. Software tools are provided to plot the configuration of the mitral annulus on multiphase CT as the heart changes shape throughout the cardiac cycle. The tools can be used to characterize differences in the behavior of the mitral annulus in a variety of pathologic conditions that may include (1) primary mitral valve disease (i.e., abnormality of the mitral valve leaflets leading to mitral regurgitation); (2) non-ischemic secondary (functional) mitral valve disease (i.e., dilatation of the left ventricle not caused by a heart attack that leads to mitral annular dilatation and associated mitral regurgitation); and (3) ischemic secondary (functional) mitral valve disease (i.e., a heart attack which leads to dilatation of the left ventricle and tethering of the mitral valve leaflets and associated mitral regurgitation).

As described above, 3D printing techniques to construct patient-specific heart models of mitral valve disease and fluid dynamics for simulation of valve prostheses deployment can be provided using the present systems and methods. The reports described above may also be used to print 3D models of the various pathologic conditions illustrated in the acquired images. These models for actual deployment of catheter directed mitral valve prostheses, to evaluate which valve prostheses fit best in the various disease models, or to determine preferred measurement techniques for predicting adequate valve fit. Furthermore, the above described reports may be used to determine animal models with different types of mitral valve diseases (e.g. primary MR, secondary MR) to provide in vivo confirmation of mitral valve match and to assess longer-term fit adequacy as the heart remodels or to develop new animal models that simulate the various mitral valve disease states.

The present disclosure provides systems and methods to utilize planning tools, including finite element analysis, to improve prosthesis selection based on patient specific anatomy to minimize the risk of the procedure. For example, finite element analysis techniques may be used to model the actual forces involved in expansion of valve prostheses, to create patient specific models of the various mitral valve disease states, or to combine the valve models and the patient specific models to simulate the actual mechanics of valve deployment and the resulting (initial) effects of the procedure.

The present disclosure has described one or more preferred embodiments, and it should be appreciated that many equivalents, alternatives, variations, and modifications, aside from those expressly stated, are possible and within the scope of the invention.

1. A method for automatically generating a treatment report comprising:
acquiring a image data from a region of a patient at least including a heart of the patient;
generating patient metric data from the imaging data, wherein the patient metric data indicate a plurality of patient-specific parameters associated with anatomy of the heart of the patient;
generating treatment plan data by processing the patient metric data, wherein the treatment plan data indicate an optimal treatment for the patient based on the anatomy of the heart of the patient; and
generating a report indicating a desired treatment for the patient based on analyzing the treatment plan data.

2. The method of claim 1, wherein the step of acquiring the image data comprises preforming a multiphase CT scan of the patient.

3. The method of claim 1, wherein the plurality of patient-specific parameters includes at least one of a circumference of a mitral annulus, a variation in circumference of the mitral annulus, an intercommissural (IC) distance, or a septal-to-lateral (SL) distance.

4. The method of claim 3, wherein generating the treatment plan data includes comparing at least one of the patient-specific metrics to a first threshold value.

5. The method of claim 4, wherein generating the report includes selecting a mitral valve prosthesis if the variation of the circumference is greater than the first threshold value.

6. The method of claim 3, wherein generating the treatment plan data includes calculating a ratio between the IC distance and the SL distance and comparing the ratio to a second threshold value.

7. The method of claim 6, wherein generating the report includes indicating a reduction of the annulus of the patient when the ratio between the IC distance and the SL distance is greater than the second threshold value.

8. The method of claim 6, wherein generating the report includes indicating a reduction of a posterior of the annulus of the patient when the ratio between the IC distance and the SL distance is less than the second threshold value.

9. The method of claim 8, wherein the report indicates one or more anchors to be used to reduce the posterior of the annulus of the patient.

10. The method of claim 1, wherein the treatment plan data indicates that the patient should be treated by placing a mitral valve prosthesis and further indicates parameters associated with an optimal mitral valve prosthesis for the patient.

11. The method of claim 10, wherein the parameters associated with the optimal mitral valve prosthesis for the patient include parameters for selecting a mitral valve prosthesis from a plurality of available mitral valve prostheses.

12. The method of claim 10, wherein the parameters associated with the optimal mitral valve prosthesis for the patient include parameters for additively manufacturing a mitral valve prosthesis specific for the anatomy of the heart of the patient.
